(19) United States
(12) Patent Application Publication
(10) Pub. No.: US 2005/0010159 A1
(43) Pub. Date: Jan. 13, 2005

(54) Csf physiologic controller

(76) Inventors: Sanford Reich, Providence, RI (US); James E. Sluetz, N. Attleboro, MA (US)

Correspondence Address: NIELDS & LEMACK, 176 EAST MAIN STREET, SUITE 7, WESTBORO, MA, 01581, US

(21) Appl. No.: 10/500,281

(22) PCT Filed: Jan. 2, 2003
(86) PCT No.: PCT/US03/00094

Related U.S. Application Data
(60) Provisional application No. 60/345,431, filed on Jan. 4, 2002.

(51) Int. Cl. A61M 5/00

(52) U.S. Cl. 604/8

(57) ABSTRACT

The Cerebrospinal Fluid (CSF) Physiologic Controller is an implantable active battery-operated device that is microprocessor controlled via algorithms stored in its memory. It is a multi mode drainage system that contains at least two flow paths: a low resistance flow path for when the patient is in the supine or substantially supine position and a flow path containing a programmable variable check valve to prevent over-drainage when the patient is in the upright or substantially upright position.

This application claims priority of Provisional Application Ser. No. 60/345,431 filed Jan. 4, 2002, the disclosure of which is hereby incorporated by reference.

### BACKGROUND OF THE INVENTION

The human skull is primarily occupied by brain tissue and the supporting blood vessels. About ten percent of this volume is clear fluid with small amounts of dissolved protein, sugar and salts. This fluid is known as cerebrospinal fluid (CSF). This CSF fluid cushions the delicate brain and spinal cord tissues from injuries and maintains the proper balance of nutrients and salts around the central nervous system.

A system of four interconnecting cavities, known as ventricles, in the brain provide pathways through which the CSF circulates from deep within the brain, around the spinal column, and over the surfaces of the brain. CSF is continually being created. In fact, about three to five times the volume contained in the skull at any point in time is produced on a daily basis.

Hydrocephalus is an abnormal accumulation of CSF in the ventricles. Hydrocephalus can be present at birth (congenital), acquired as a result of brain trauma, or can occur in adults in a condition known as normal pressure hydrocephalus.

Normally, almost all of the CSF is absorbed into the bloodstream, thus maintaining the delicate balance between CSF production and absorption. This fluid system becomes unbalanced when the rate of CSF production in the ventricles is greater than the rate of CSF absorption into the bloodstream. The excess fluid causes increased intraventricular pressure (IVP). A high level of pressure for any sustained period can lead to serious complications.

The most common treatment for hydrocephalus is shunt therapy; a surgical procedure in which a hydrocephalus valve system is usually implanted while the patient is under general anesthesia. In this commonly used procedure, a small hole is made in the skull and the protective membrane overlaying the brain. An incision is made in the abdomen and the valve unit and associated tubing are introduced under the skin between the scalp and the abdominal incisions. Usually one ventricular cannula is inserted into the lateral ventricle and connected to the drainage tube, which is inserted in the abdominal cavity. The drainage cannula may also be introduced through a neck incision and passed through various blood vessels until the tip of the cannula is positioned in the right atrium of the heart. This system is intended to allow CSF from the ventricle to travel through the implanted tubes into either the abdominal cavity or the heart, where it is then absorbed into the bloodstream.

Under-drainage, in which the fluid is not removed quickly enough, is a common problem of the shunt system. Sometimes under-drainage may be due to the shunt cannula breakage or disconnection. Valve blockage is relatively uncommon. This breakage or disconnection disrupts the new path made for the CSF and causes increased pressure in the ventricles. Rapid increase in IVP may result in loss of consciousness, and emergency treatment is required. However, in most cases, the onset is more gradual, and can follow a minor illness, such as a cold. Headaches increase in frequency and severity, often worse upon waking in the morning. Vomiting and dizziness may also occur, and sometimes there may be other symptoms, which vary from patient to patient.

Over-drainage, in which the shunt allows CSF to drain from the ventricles more quickly than it is being produced, is also a common problem in shunt therapy. If this happens suddenly, such as soon after the shunt is inserted, then the ventricles of the brain may collapse, tearing delicate blood vessels on the outside of the brain and causing a hemorrhage. This can be trivial or it can cause symptoms similar to those of a stroke. The blood may have to be removed, and in some cases, if this is not done, it may be the cause of epilepsy later. If the over-drainage is more gradual, the ventricles collapse gradually to become slit-like. This often interferes with the function of the shunt, causing the opposite problem, high IVP. Unfortunately, the slit ventricles may not always increase is size, resulting in headache and vomiting.

The symptoms of over-drainage can be very similar to those of under-drainage with an important difference. With over-drainage, headaches often become worse getting up from a supine (horizontal) position. This is because the change in position causes excessive drainage to occur, since gravity forces more CSF to drain. With under-drainage, headaches caused by high IVP often become worse on waking in a supine position. This is because little CSF is drained in the horizontal position, causing an increase in IVP. The best way to distinguish between these two conditions is to monitor the IVP over 24 hour periods.

The drainage rate of the shunts varies depending on the patient's relative position. In an upright position, an increased rate of CSF flow is generated, since gravity serves to create siphoning pressure, which will aid in the drainage process. In the supine, or horizontal, position, drainage is caused solely by the imbalance of pressure. Current shunt therapy devices are not designed to effectively treat over-drainage. These devices still maintain a large negative IVP (over-drainage) when the patient is in the upright position. A change of valve to a higher pressure cannot be relied upon to cure it, though it appears to do so in some cases. Anti-siphon devices, which consist of a small button inserted into the shunt tubing, may sometimes solve the problem. Some shunts have these built-in, but neurological opinion varies as to whether they should be used. To change a valve pressure, surgery is necessary to remove the valve and insert another. A relatively new shunt, the ‘programmable’ or adjustable shunt, is intended to allow adjustment of the working pressure of the valve without surgery. This valve contains magnets that allow the valve pressure setting to be altered by a transcutaneous magnetic field placed over the scalp. This is useful where the need for a valve of a different pressure arises, but the adjustable valve is no less prone to the over-drainage issue than any other and it cannot be used to treat this condition.

Normal pressure hydrocephalus (NPH) is an accumulation of cerebrospinal fluid that causes the ventricles to become enlarged with a return to normal pressure. The name of this condition is misleading, however, because some patients have fluctuations of IVP from high to normal to low. In most cases of NPH, it is not clear what causes the CSF pathways to become blocked.

Normal intraventricular pressure (IVP) is between 10-15 mm Hg in the supine position and −5 mm Hg in the upright position.

Adult-onset normal pressure hydrocephalus described those cases that occur in older adults (age 50 and older). The majority of the NPH population is 60 years or older. In the majority of cases of NPH, the cause is unknown. In some cases, NPH can develop as a result of a head injury, cranial surgery, subarachnoid hemorrhage, meningitis, tumor or cysts, as well as subdural hematomas, bleeding during surgery and other infections. The syndrome of NPH is usually characterized by complaints of gait disturbance (difficulty walking), mild dementia and impaired bladder control.

Hydrocephalus is often classified as either communicating or non-communicating. In the former, the problem is usually failure to absorb the CSF at the end of the system, whereas in the latter, there is blockage of the CSF pathways within the ventricular system.

In summary, the limitations of the current implantable shunt technologies are as follows. The current shunt systems use passive components such as check valves to regulate the flow of CSF. These passive check valves are designed to open when a predetermined pressure drop exists across the check valve. Short-term changes, such as when the patient rises from a horizontal position to a standing position, may cause excess drainage because of the added siphoning of the vertical tubing. In the longer term, passive check valves are not able to automatically maintain normal IVP by adjusting CSF drainage if the patient experiences changes in CSF generation. Note that the selection of a pressure valve may result in a compromise between under-drainage in the supine position and over-drainage in the upright position.

The current shunt systems have no method for non-invasively measuring the CSF drained flow rate. Therefore, once installed, it is difficult to monitor the shunt's operation.

The current shunt systems cannot monitor IVP except during an invasive procedure, requiring a needle. Sustained low or high IVP may lead to serious complications.

The current shunt systems do not have the capability to monitor, store, and transmit data related to CSF flow, IVP or cannula operation.

The current invention is primarily targeted toward the adult-onset normal pressure hydrocephalus population. It is the objective of the present invention to create an apparatus to overcome all of the shortcomings listed above.

### SUMMARY OF THE INVENTION

The limitations of the current shunt therapy for hydrocephalus have been overcome by the present invention. The CSF Physiologic Controller is an implantable active battery-operated device that is microprocessor controlled via algorithms stored in its memory. It is a multi-mode drainage system that contains at least two flow paths: a low resistance flow path for when the patient is in the supine or substantially supine position and a flow path containing a programmable variable check valve to prevent over-drainage when the patient is in the upright or substantially upright position. The Controller also contains numerous diagnostic features, which enable the physician to monitor the operation of the system, as well as several key patient parameters non-invasively.

### BRIEF DESCRIPTION OF THE DRAWINGS

FIG. 1 is a flow schematic of the CSF Controller;

FIG. 2 is a view of the first embodiment of the variable check valve, located within the CSF Controller;

FIG. 3 is a graph demonstrating the check valve's performance over a range of conditions;

FIG. 4 is the preferred implantation of the CSF system in the patient;

FIG. 5 is a detailed view of the CSF Controller; and

FIG. 6 is a second embodiment of the variable pressure valve.

### DETAILED DESCRIPTION OF THE INVENTION

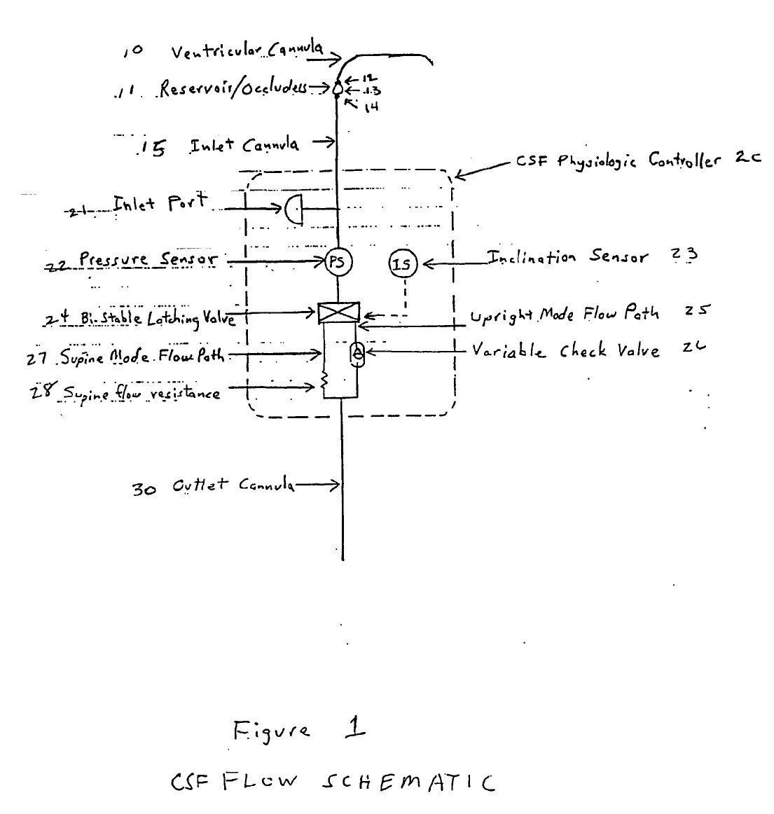

The CSF Physiologic Controller is a multi mode drainage system that contains at least two flow paths: (1) a supine mode: a low resistance flow path for when the patient is in the supine or substantially supine position and (2) an upright mode: a flow path containing a programmable variable check valve to prevent over-drainage when the patient is in the upright or substantially upright position. A bi-stable latching valve directs the CSF flow to either the low resistance path or the check valve path based on an inclination sensor within the CSF Physiologic Controller. If the inclination sensor angle is below a programmable critical angle, the bi-stable latching valve directs flow to the low resistance path. If the inclination sensor angle is equal to or above a critical programmable angle, the bi-stable latching valve directs flow to the check valve path. For purposes of illustration, a dual mode controller will be described; however, the present invention is not limited to only two modes. FIG. 1 shows the flow schematic of the CSF Controller System.

The ventricular cannula 10 is typically implanted in the ventricle of the patient's brain. It serves as the source for the CSF fluid into the system. The ventricular cannula is in fluid communication with the reservoir/occluders device 11. This device is implanted just beneath the scalp and can be actuated by pressing on the scalp. This device contains a reservoir 13 for holding CSF fluid. On either side of the reservoir is a manual blocking mechanism, known as an occluder. The one nearer to the ventricle is known as the proximal occluder 12, while the other is the distal occluder 14. These occluders allow the physician to interrupt the flow of CSF to perform a number of in-office non-invasive diagnostics.

The distal occluder 14 is in fluid communication with the inlet cannula 15, which is a tube that is in fluid communication with the CSF Physiologic Controller 20. The Physiologic Controller is preferably located below the clavicle in the pectoral area. It regulates the flow of CSF through it, and the outgoing CSF flows into the outlet cannula 30. This outlet cannula is implanted such that its distal (far) end is inserted into the peritoneal space or inserted intravenously with its distal tip in the right atrium of the heart.

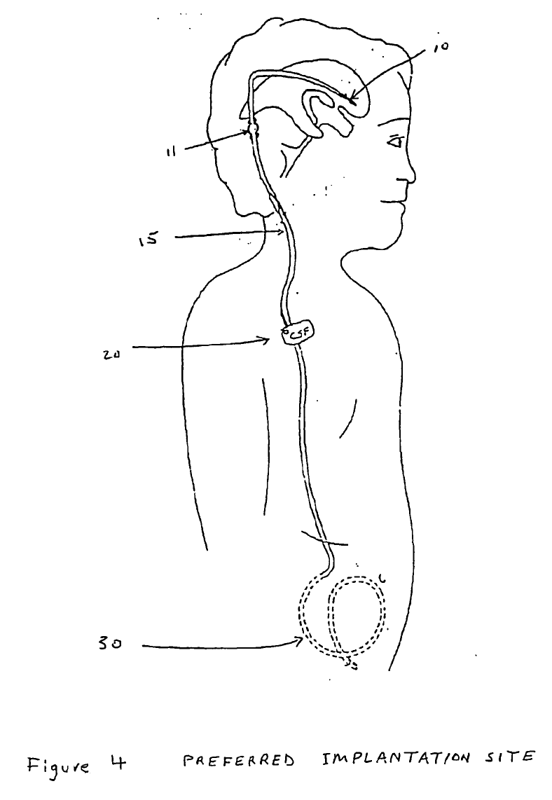

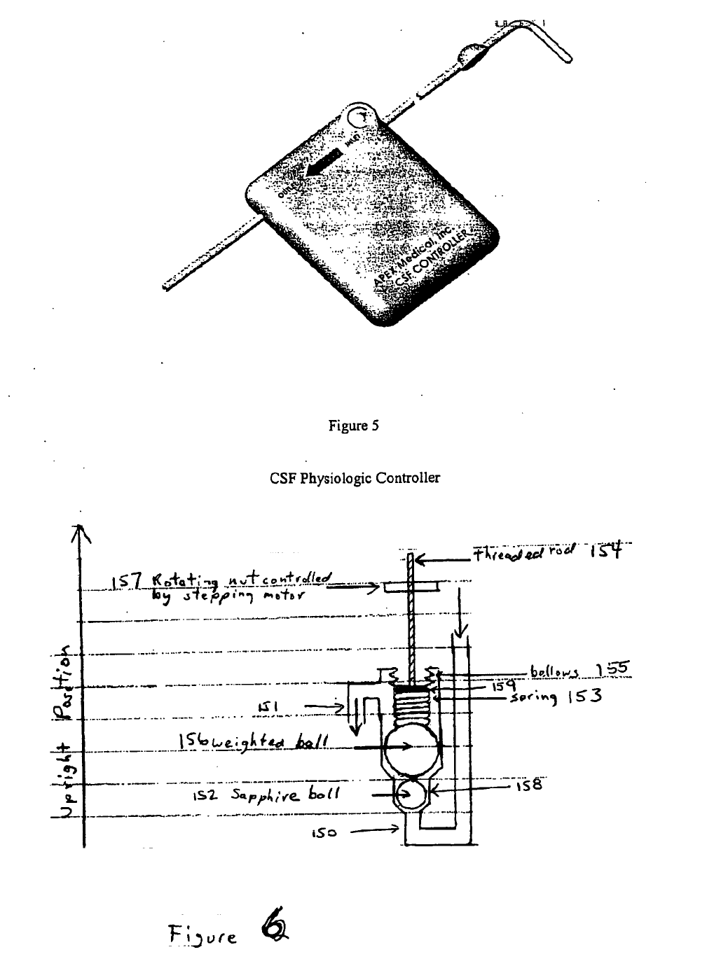

FIG. 4 shows the preferred implantation site for the CSF controller. The ventricular cannula is inserted into the ventricle of the brain with its distal end in fluid communication with the reservoir/occluder 11, which is placed just under the scalp. The inlet cannula 15 traverses the body from the scalp to the CSF Controller 20, which is located in the pectoral area of the chest. The outlet cannula 30 has its distal end placed in either the abdominal cavity or the right atrium of the heart.

Referring back to FIG. 1, the CSF Physiologic Controller 20 contains all of the mechanisms required to implement the CSF flow. The inlet port 21 is used to access fluid in the inlet cannula. The side branch, that is only used occasionally, is not part of the main fluid path so as to prevent accumulation of protein particle with the side branch. The inlet port 21 is preferably a silicone rubber septum assembly through which a doctor may insert a needle for the purpose of sampling the CSF, to inject a dye for diagnostic purposes, or inject saline to check for flow blockages. In this implementation, the port is also used to periodically calibrate a pressure sensor.

The inlet cannula flows into the pressure sensor component 22, located within the CSF controller 20. The purpose of this sensor is to determine the relative pressure of CSF at the inlet cannula. The following is for illustrative purposes only; a number of different embodiments could be used to implement the pressure sensor. In this embodiment, the pressure sensor component is actually two distinct MEMS (Micro-Electro-Mechanical Systems) absolute pressure sensing silicon elements. The two MEMS silicon pressure-sensing elements may be attached to a common vacuum. The non-vacuum sides of each are oil-coupled to the force-collecting diaphragms. The top force-collecting diaphragm is integral with a flat portion of the CSF fluid path and measures the absolute pressure in the CSF path. The lower force-collecting diaphragm is in communication with the outside bottom portion of the device and measures the absolute pressure on the outside of the device. This outside pressure sensing element measures the tissue pressure of the implanted device and closely tracks the atmospheric pressure. A mechanical guard over the outside force-collecting diaphragm protects it from mechanical forces that may produce pressure artifacts. The difference between the two absolute pressure sensors is the gauge pressure of the CSF at the inlet to the Controller. This pressure is indicative of the intraventricular pressure (IVP). In the supine position, this reading is roughly equivalent to the IVP. In the upright position, this reading is the IVP plus the siphon pressure created by the shunt. By using the inclination sensor, it is possible to determine the actual IVP of the patient regardless of the inclination angle. In normal operation, the pressure sensor monitors the intraventricular pressure (IVP) not continuously, but periodically, for example, every 2-5 minutes. These readings can be stored in the Controller's memory. Using the telemetry capability of the Controller to download the information to the external programmer, the physician may review daily changes in IVP to diagnostic purposes. For example, the physician may choose to do this when a patient complains of headaches. The CSF Controller can sample the pressure sensor at any time to determine the IVP, as measured at the input to the Controller.

The inclination sensor 23 is a gravity-detecting sensor that is used to determine the patient's inclination angle. It is used to control the multi mode CSF Physiologic Controller. This sensor also detects patient activity, such as when the patient is resting or moving about. Both the inclination and activity functions may be utilized to control the bi-stable latching valve 24.

The bi-stable latching valve 24 directs the CSF flow to the low resistance, supine mode path 27 when the inclination sensor 23 indicates that the patient is in a supine or substantially supine position, or the upright mode flow path 25 when the inclination sensor indicates that the patient is in an upright position.

The supine mode flow path 27 includes a supine flow resistance 28, which is designed to prevent against under-drainage and keep the IVP within the normal upper limit of 15 mm Hg. In this embodiment, the supine flow resistance is simply the resistance of the cannula in the supine mode flow path. The upright mode flow path 25 provides a variable high resistance flow path that is designed to prevent over-drainage. The variable high resistance flow path is provided by a variable check valve 26 whose cracking pressure is automatically adjusted based on the inclination angle.

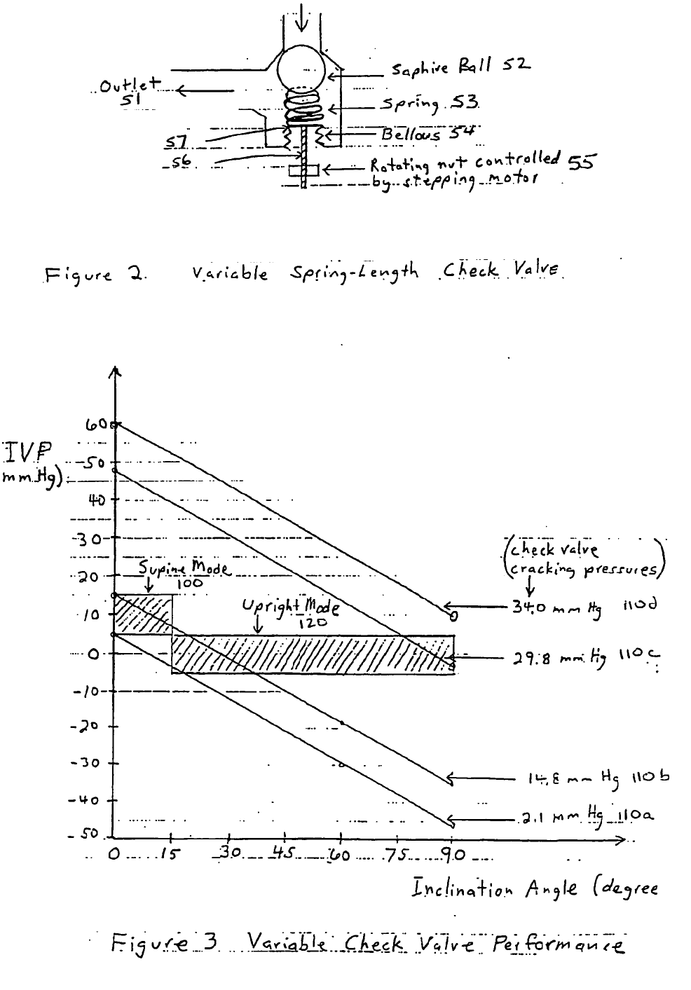

FIG. 2 shows a suitable design for a variable check valve. This diagram is for illustrative purposes only, and the check valve is not limited to a particular valve embodiment. The CSF flow originates at the inlet 50. A ball 52 serves to block the CSF from passing from the inlet 50 to the outlet 51. The ball 52 is preferably constructed of a material not deleterious to the application, such as sapphire, which does not interact with the cerebrospinal fluid. The ball is preferably small in diameter in order to ensure the best seal when the ball is resting on the inlet 50. For CSF to pass to the outlet, the pressure of the CSF at the inlet 50 must exceed the pressure exerted by the spring 53. The point at which this occurs is known as the cracking pressure. At this point, the ball will rise and allow the CSF to flow through the inlet 50 and onto the outlet. The spring 53 is located between the sapphire ball 52 and a horizontal platform 57. This horizontal platform can be moved both up and down by rotating screw 56. As the horizontal platform is moved up, the cracking force increases. Likewise, as the horizontal platform is lowered, the cracking force decreases. Bellows 54 covers the horizontal platform to insure that the valve is fluid-tight. The rotating screw 56 is controlled by a nut 55, which in turn is controlled by a stepping motor (not shown). In this embodiment, the stepping motor controls the nut as a function of the patient's inclination angle, as determined by the inclination sensor 23.

FIG. 6 shows a second, preferred embodiment of the variable check valve assembly. The CSF flow originates at the inlet 150 at the base of the valve. A small ball 152, again preferably sapphire, sits atop the inlet 150, forming a seal. Sapphire is used because it does not interact with the CSF. The ball is preferably small in diameter in order to ensure the best seal when the ball is resting on the inlet 150. This ball, which is held in place by valve housing 158, serves to block the CSF from passing from the inlet 150 to the outlet 151. A weighted ball 156, preferably made of tantalum because of its high density and its inertness, is located between the sapphire ball 152 and the spring 153 and rests against the valve housing 158. In this illustration, the weighted ball is shown to be larger than the sapphire ball. While this is the preferred implementation, the invention is not subject to this limitation. In order for the sapphire ball to be unseated, the pressure of the CSF at the inlet 50 must exceed the pressure exerted by the spring 153 plus the downward force of the weighted ball 156. The point at which this occurs is known as the cracking pressure. Note that when the patient is in the upright position, the downward force of the weighted ball 156 on the sapphire ball is equal to its weight. However, in the vertical position, the weighted ball exerts no additional force on the sapphire ball, as the gravitational force will be against the valve housing 158. Thus the force exerted by the weighted ball 156 on the sapphire ball can be expressed as the weight of the ball multiplied by the sine of the inclination angle of the patient, where an inclination angle of 0° signifies a supine position and an inclination angle of 90° indicates a fully upright position. The spring 153 is located between the sapphire ball 152 and a horizontal platform 159. This horizontal platform can be moved both up and down by rotating threaded rod 154. As the horizontal platform 159 is moved toward the sapphire ball 152, the force of the spring 153 increases, therefore the cracking force increases. Likewise, as the horizontal platform 159 is moved away from the sapphire ball, the force of the spring decreases, therefore the cracking force decreases. Bellows 155 covers the horizontal platform 159 and seals to the valve housing 158 to insure that the valve is fluid-tight. The threaded rod 154 is controlled by a rotating nut 157, which in turn is controlled by a stepping motor (not shown). The stepping motor is controlled by the microprocessor in the controller. In this embodiment, the stepping motor controls the nut, which turns the threaded rod, and causes the horizontal platform to move. This adjustment is carried out to set the correct cracking pressure when the patient is in the upright position. The cracking pressure is made up of two components, a fixed component, which is set using the spring force and a gravitational variable component, which is determined by the weighted ball. The variation in cracking pressure required as the patient changes inclination are mostly handled by the variation in the downward gravitational force of the weighted ball, thereby significantly reducing the power required to maintain a stable intraventricular pressure over a range of inclination angles.

Those skilled in the art will appreciate that the gravitational component of the valve assembly could be in fluid communication with a separate inlet from the inlet that the fixed component is in fluid communication with, in which case the gravitational component and fixed component would function in series.

FIG. 3 graphically illustrates the operation of the check valve. This particular graph is done for purposes of illustration, and the invention is not limited to this functionality. This graph shows intraventricular pressure graphed as the vertical axis, with patient's inclination angle as the horizontal axis. For clarity, 0 degrees denotes a person in the completely horizontal position, while 90 degrees is a patient in the fully upright position. In this example, the siphon length was 62 cm. Four diagonal lines 110a-d show lines of constant check valve cracking pressure. As an example, if the check valve cracking pressure were held constant at 2.1 mm Hg, as in line 110a, the IVP would be 5 mm Hg in the supine position. The IVP would decrease as the inclination angle increased, reaching a value of about −45 mm Hg when the patient is fully upright. Similarly, line 110d illustrates that for a cracking pressure of 34.0 mm Hg, the IVP is 60 mm Hg when the patient is fully supine and about 10 mm Hg when the patient is completely upright. Lines 110b and 110c show similar trends at 14.8 mm and 29.8 mm, respectively. The shaded area, supine mode 100, denotes the desired IVP when the patient is in the supine or substantially supine position. As used herein, substantially supine is defined as less than about 15 degrees of inclination (accordingly, substantially upright is an inclination angle greater than about 15°). In the present invention, this result is achieved using the low resistance supine mode flow path 27 in the CSF. Once the patient's inclination angle exceeds about 15 degrees, the CSF uses the upright mode flow path 25. In this mode, the desired IVP range is shown in the shaded area, upright mode 120. At 15 degrees, the valve cracking pressure is between 2.1 mm and 14.8 mm in order to achieve an IVP of −5 to 5 mm Hg. As the patient becomes more upright (i.e., the inclination angle approaches 90°), the cracking pressure increases in order to maintain the desired IVP. When the patient is fully upright, the cracking pressure is about 29.8 mm Hg in order to maintain the proper IVP.

These graphs can be generated using the preferred embodiment of the programmable cracking pressure valve described in FIG. 6. While the patient is in the upright position, the spring tension is adjusted such that the IVP is between 5 and −5 mm Hg. As the patient reclines toward horizontal, a lower cracking pressure is needed to maintain the desired IVP range. The gravitational component of the cracking pressure, which is contributed by the weighted ball, is reduced as the patient reclines, thereby lowering, without any use of battery power, the cracking pressure of the valve. In this way, the siphon pressure created by the fluid contained within the length of the inlet cannula from the brain to the controller is roughly counterbalanced by the effect of the weighted ball. By using the combination of the programmable spring force and the weighted ball, it is therefore possible to maintain the IVP within the desired range as shown in FIG. 3.

In addition to the elements described above, which are part of the flow paths, there is a microprocessor-based subsystem internal to the CSF Controller. This subsystem preferably comprises a microprocessor, its associated memory, a Real Time Clock, a wireless transceiver and other essential electronics. An internal battery powers this subsystem. The microprocessor is responsible for monitoring and controlling many of the operations enumerated above, such as monitoring the inclination sensor, adjusting the check valve cracking pressure in response to changes in inclination, and monitoring the pressure sensor. The microprocessor is also capable of receiving commands and returning status to the external programmer via the wireless transceiver. The memory is used to store data requested by the external programmer, such as pressure readings, inclination angle, and time. This data can be transmitted back to the external programmer as requested, via the wireless transceiver. The Real Time Clock is used to enable the Controller to perform certain diagnostics at specific times.

In conjunction with the CSF Controller, there is an accompanying external programmer. This programmer is typically used by a physician, and is used to program critical parameters in the CSF Controller, retrieve stored information from the CSF Controller, and perform other types of communication with the CSF Controller. The external programmer can also be used to perform a number of diagnostic procedures in conjunction with the CSF Controller. The external programmer permits the physician to program the desired critical angle at which the CSF Controller switches from supine to upright mode. The external programmer can also be used to preset the spring tension for the preferred embodiment of the variable cracking pressure valve, shown in FIG. 6. This adjustment is used to create the patient unique version of FIG. 3. The external programmer also contains a MEMS based barometer that is used to calibrate the tissue pressure sensor in the CSF Controller.

The external programmer can take many different physical forms. It preferably comprises the following set of components:
a processor unit to perform the necessary algorithms and calculations;
an internal memory to store data received from the controller, and other relevant information;
a data input device to accept input from the physician;
a data output device to display data to the physician;
and a communication port to transmit information to the CSF Controller.

The external programmer can be a custom developed apparatus, or can be an existing device, such as a Palm™ handheld or laptop computer. In the scenario where a Palm™ handheld is used, the criteria above are met as follows. The processor unit and internal memory are standard elements of the Palm™ handheld. The data input device is the touch screen of the device, or the optional keyboard. The data output device is also the touch screen. Lastly, the communication to the CSF Controller is performed by an optional wireless module that can be connected to the Palm™ handheld.

1. A system for regulating the flow of cerebrospinal fluid from the brain of an individual comprising an implantable controller adapted to be in fluid communication with said cerebrospinal fluid and having first and second drainage paths, wherein said controller directs the flow of said cerebrospinal fluid into said first or second drainage paths in response to the inclination of said individual.

2. The system of claim 1, wherein said first drainage path is a supine flow path, and wherein said controller directs the flow of said fluid into said supine flow path when said individual's inclination is supine or substantially supine.

3. The system of claim 1, wherein said second drainage path is an upright flow path, and wherein said controller directs the flow of said fluid into said upright flow path when said individual's inclination is vertical or substantially vertical.

4. The system of claim 1, further comprising an inclination sensor for sensing the inclination of said individual, and wherein said controller is responsive to said inclination sensor.

5. The system of claim 4, further comprising a bi-stable latching valve, and wherein said controller directs the flow of said fluid by actuating said latching valve to allow for fluid communication with said first or said second drainage paths.

6. The system of claim 2, wherein said supine flow path comprises a passive low resistance flow path.

7. The system of claim 6, wherein said passive low resistance flow path maintains a maximum intraventricular pressure of about 15 mm Hg.

8. The system of claim 1, further comprising a programmable variable check valve in said second flow path, wherein the cracking pressure of said check valve is modified based on the inclination angle of said individual.

9. The system of claim 8, wherein said cracking pressure is continually modified to maintain a relatively stable intraventricular pressure for a range of inclination angles.

10. The system of claim 9, wherein said stable intraventricular pressure is between 5 and −5 mm Hg.

11. The system of claim 1, wherein said controller implanted in said individual further comprises:
an inlet connection;
an outlet connection spaced from said inlet connection;
an inlet cannula with a distal and proximal end, wherein said distal end of said inlet cannula is located near the ventricle of the brain and said proximal end of said inlet cannula is connected to said inlet connection of said controller; and
an outlet cannula with a distal and proximal end, wherein the location of said distal end of said outlet cannula is selected from the group consisting of the peritoneal space and the right atrium of the heart, and said proximal end of said outlet cannula is connected to said outlet connection of said controller.

12. A method of regulating the flow of cerebrospinal fluid from the brain of an individual, comprising:
determining the inclination angle of said individual; and
directing the flow of cerebrospinal fluid through one of two flow paths in response to said determined inclination angle.

13. The method of claim 12, wherein said inclination angle is determined by an implantable inclination sensor.

14. The method of claim 12, wherein one of said flow paths is a substantially supine mode flow path and is a passive low resistance path.

15. The method of claim 12, wherein one of said flow paths is an upright mode flow path further comprising a programmable variable pressure check valve, wherein the cracking pressure of said check valve is modified based on said inclination angle of said individual in order to maintain relatively stable intraventricular pressure.

16. The method of claim 15, wherein said stable intraventricular pressure is between −5 and 5 mm Hg.

17. An implanted system for maintaining a stable intraventricular pressure of an individual by regulating the flow of cerebrospinal fluid from the brain of said individual, comprising:
a variable cracking pressure valve assembly in fluid communication with said cerebrospinal fluid, wherein the cracking pressure of said valve assembly comprises a gravitational component, responsive to changes in inclination angle and a fixed component, wherein said fixed component is responsive to actions from a microprocessor based controller system; and
said microprocessor-based controller system, capable of adjusting said fixed component of said cracking pressure.

18. The system of claim 17, further comprising a weighted ball contained within said valve assembly, wherein said gravitational component is created by said weighted ball.

19. The system of claim 17, wherein said microprocessor adjusts said fixed component to create a stable intraventricular pressure profile over a range of inclination angles.

20. The system of claim 17, further comprising a spring having an end in contact with a movable element contained within said valve assembly, wherein said fixed component is created by the bias of said spring and said fixed component is adjusted by changing said bias of said spring by moving said movable element.

21. The system of claim 17, said valve assembly further comprising:
an inlet port adapted for the intake of said cerebrospinal fluid;
an outlet port spaced from said inlet port;
a first ball positioned in said valve assembly, between an open position in which said inlet port is in fluid communication with said outlet port and a sealed position in which said ball seals said inlet port;
a second ball positioned in said valve assembly, biased against said first ball, whereby the force needed to overcome said bias and allow said first ball to move from said sealed position to said open position is a function of the gravitational force on said second ball;
a movable element, with movement responsive to said microprocessor-based subsystem; and
a spring, biased between said second ball and said movable element, whereby said movable element can be moved to cause a change in bias of said spring by said microprocessor-based subsystem.
